(19) United States
(12) Patent Application Publication
(10) Pub. No.: US 2024/0189615 A1
(43) Pub. Date: Jun. 13, 2024

(54) METHOD AND SYSTEM FOR PRODUCING APOPTOTIC MONONUCLEAR CELLS

(71) Applicant: MACO PHARMA, Mouvaux (FR)

(72) Inventors: Sylvie LEFEBVRE, Tourcoing (FR); Chryslain SUMIAN, Kortrijk (BE)

(21) Appl. No.: 18/268,413

(22) PCT Filed: Dec. 16, 2021
(86) PCT No.: PCT/EP2021/086083

§ 371 Date: Dec. 8, 2023

(30) Foreign Application Priority Data
Dec. 21, 2020 (FR) FR2013771

(51) Int. Cl. A61N 5/06; A61K 35/17

(52) U.S. Cl. CPC A61N 5/06; A61K 35/17; A61N 2005/0661

(57) ABSTRACT

The invention relates to a method and apparatus for producing apoptotic mononuclear cells contained in a fraction of peripheral blood from a donor. The method includes subjecting the peripheral blood fraction to ultraviolet irradiation having a wavelength of between 200 nm and 320 nm. The irradiation is arranged to induce a difference between the degree of apoptosis of the irradiated cells and that of the non-irradiated cells of greater than 15%, 48 hours after the irradiation, se as to obtain apoptotic mononuclear cells capable of modulating the immune response in a patient.

The invention relates to a method, an irradiation apparatus and a system for producing apoptotic mononuclear cells contained in a peripheral blood fraction from a donor, and to the use of these cells for the treatment of pathologies associated with immune system dysfunction.

The invention applies to the fields of cell therapy and immunotherapy, particularly extracorporeal photo-immunotherapy.

Photopheresis, also known as extracorporeal photochemotherapy (ECP), is a cell therapy technique that emerged in the 1980s following Edelson's work in the treatment of cutaneous T-cell lymphoma (CTCL) (Edelson et al., 1987). It involves taking blood from a patient, separating the mononuclear cells (MNC) from the patient's blood by centrifugation, treating these cells with ultraviolet A (UVA) irradiation in the presence of a photoactivatable intercalating agent called 8-methoxypsoralen (8-MOP), and then reinjecting the activated cells into the patient. The cells are treated ex vivo.

ECP has proved its worth in the treatment of pathologies linked to immune system dysfunction, either in an autologous situation (specific to the patient), for example for tumour or autoimmune pathologies, or in an allogeneic situation (donor-recipient relationship) in the case of a solid organ or haematopoietic stem cell (HSC) transplant that may be associated with a major complication such as graft versus host disease (GvHD).

The safety and efficacy achieved with this therapy are now prompting clinicians to broaden the therapeutic scope of ECP, particularly in the treatment of autoimmune diseases (Adamski et al., 2015).

The mode of action of ECP is not completely understood. However, it is accepted that 8-MOP passes through the cell membrane and intercalates between the two DNA strands. After UVA irradiation, 8-MOP is activated, leading to a photoaddition process (Jeantet, 2004). This process blocks DNA replication and transcription, leading to cell proliferation arrest and apoptosis. Among the mononuclear cells treated during ECP, the T lymphocyte population is the one most affected by apoptosis (Heshmati, 2014). Apoptosis is a programmed cell death that is highly regulated by the organism, the other type of cell death being necrosis, a so-called “accidental” death occurring after an environmental disturbance.

Once reinjected into the patient, ECP-treated T lymphocytes undergo apoptosis and are phagocytosed by antigen-presenting cells (APCs), also known as dendritic cells, activating the immune system either towards an immunotolerant response (GvHD, transplant rejection) or towards an immunoactive response (cutaneous T lymphoma).

More specifically, ECP stimulates the differentiation of monocytes into dendritic cells, which then phagocytose the apoptotic bodies of T lymphocytes. The role of dendritic cells is to present at their surface the peptides from the phagocytosed element in order to signal the presence of a foreign body or to activate specialised T cells and induce an immune response associated with the release of pro-inflammatory or tolerogenic cytokines.

Two types of ECP procedure are currently used, depending on the institution.

The “closed” or “on-line” technique proposed by Therakos is a technique in which the stages of sampling, cell separation, processing and reinjection are carried out using a single system, in a single operation and in a closed system (Cellex system).

The “open” or “off-line” technique proposed in particular by Maco Pharma is a technique in which the sampling/separation and processing stages are carried out using different equipment, requiring several operations.

These two techniques, however, are still relatively long and complex to implement, and there is a constant need to simplify ECP.

Document WO 2020/139495 proposes various systems and methods for performing ECP on small volumes (less than 500 ml), in particular by reducing the number of devices to be handled. However, these systems and methods still require the use of 8-MOP and UVA irradiation.

To offset the possible risk of developing malignant skin tumours after using psoralen activated by UVA irradiation, WO 2015/162279 proposes using 5-aminolevulinic acid (5-ALA), a precursor of protoporphyrin X, instead of 8-MOP. However, the technique involves the same multiple steps as for ECP with 8-MOP/UVA.

WO 2017/005700 discloses an apoptotic agent-free alternative method to the ECP method for obtaining activated monocytes stimulated to differentiate into APCs. The method involves subjecting a patient blood sample comprising monocytes to a shear force by circulating the blood sample through a flow chamber.

Application WO 2016/170541 describes cell preparations comprising an enriched and pooled population of apoptotic mononuclear cells. Mononuclear cells from different donors are induced into apoptosis by incubation with methylprednisolone and then subjected to gamma irradiation to suppress cross-reactivity between the cells from different donors. UV irradiation is cited as an alternative to gamma irradiation. These cell preparations are useful for the treatment of immune, anti-inflammatory or auto-immune disease, and in particular for the treatment of graft versus host disease.

The publication by a team of dermatologists, Tuchinda, Chanisada, et al. “Comparison of broadband UVB, narrowband UVB, broadband UVA and UVA1 on activation of apoptotic pathways in human peripheral blood mononuclear cells.” Photodermatology, photoimmunology & photomedicine 23.1 (2007): 2-9, concerns the induction of apoptosis by UVA or UVB radiation in peripheral blood mononuclear cells previously washed, i.e. free of plasma and red blood cells. For this, caspase activation was measured as a function of the type and dose of irradiation of the cells. It is concluded that all UV radiation induces apoptosis, and it is suggested that apoptosis could play a role in certain inflammatory skin diseases that respond to UVB, such as psoriasis.

J. Narbutt, et al. in the publication “The effect of repeated exposures to low-dose UV radiation on the apoptosis of peripheral blood mononuclear cells.” Archives of dermatology 145.2 (2009): 133-138, compared the effect of repeated exposure to low-dose UVB (with a lamp emitting 54% UVB and 46% UVA) or simulated solar radiation (with a lamp emitting 4% UVB and 96% UVA) of the whole body or only part of the body of a volunteer on their peripheral blood mononuclear cells. On the basis of this study, it was concluded that UVA, but not UVB, induces lymphocyte apoptosis via photosensitised oxygen radicals.

Lastly, Buchele, Vera, and Holger Hackstein. “A simplified extracorporeal photopheresis procedure based on single high-dose ultraviolet A light irradiation shows similar in vitro efficacy.” Transfusion (2020) proposes to perform ECP without 8-MOP by subjecting blood mononuclear cells to high-dose UVA irradiation (5 J/cm²).

The invention proposes a simpler and faster method for mimicking the effects of ECP on mononuclear cells in the absence of a photoactivatable agent.

To this end and according to a first aspect, the invention proposes a method for producing apoptotic mononuclear cells contained in a peripheral blood fraction of a donor, said peripheral blood fraction having a plasma content of between 30 and 50%, said method comprising the step of subjecting said blood fraction to ultraviolet irradiation having a wavelength of between 200 nm and 320 nm, said irradiation being arranged to induce a difference between the rate of apoptosis of the irradiated cells and that of the non-irradiated cells of greater than 15%, 48 hours after irradiation, in particular 24 hours after irradiation, so as to obtain apoptotic mononuclear cells capable of modulating the immune response in a patient.

According to a second aspect, the invention relates to an irradiation apparatus for implementing the method according to the first aspect, the apparatus comprising an ultraviolet light source emitting radiation having a wavelength of between 200 and 320 nm, and a control unit configured to subject the peripheral blood fraction containing the mononuclear cells to a predetermined dose of ultraviolet irradiation to induce a difference between the rate of apoptosis of the irradiated cells and that of the non-irradiated cells of greater than 15%, 48 hours after irradiation.

According to another aspect, the invention consists of a system for producing apoptotic mononuclear cells contained in a peripheral blood fraction of a donor according to the method as per the first aspect, said system comprising
(a) on the one hand an irradiation container intended to receive a peripheral blood fraction containing mononuclear cells, said irradiation container being permeable to rays having a wavelength of between 200 and 320 nm, and
(b) on the other hand an irradiation apparatus according to the second aspect of the invention.

Another aspect of the invention relates to the use of the apoptotic mononuclear cells obtained by the method according to the first aspect, for the treatment of pathologies associated with immune system dysfunction.

Further features and advantages will be described below.

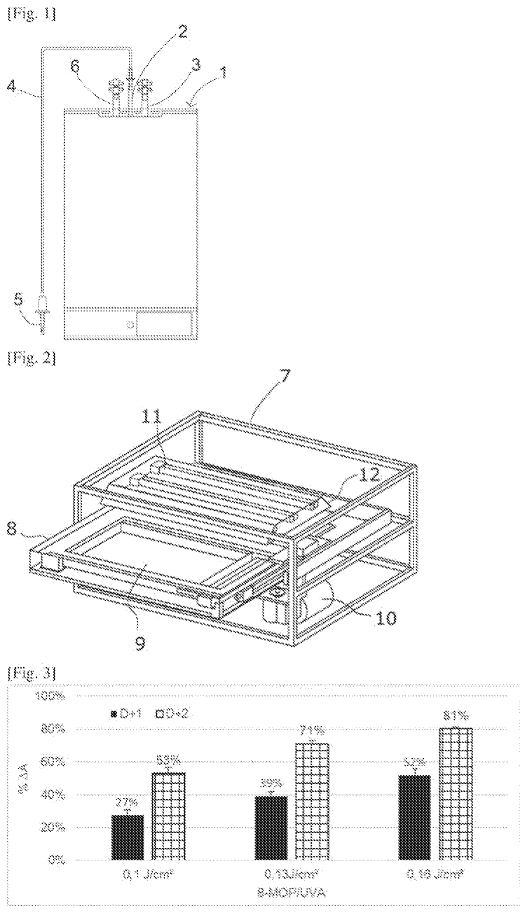

FIG. 1 shows a schematic view of an irradiation bag of a system according to the invention.

FIG. 2 shows a schematic view of an irradiation apparatus of a system according to the invention.

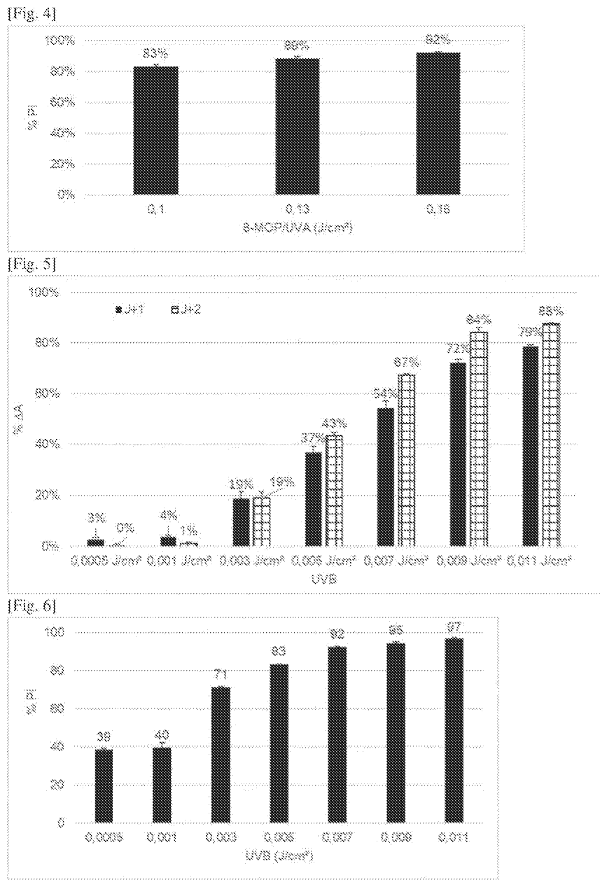

FIG. 3 shows the delta-apoptosis (%) of JURKAT cells irradiated with UVA in the presence of 8-MOP at different doses (J/cm²), 1 and 2 days after irradiation.

FIG. 4 shows the inhibition of proliferation (%) of JURKAT cells irradiated with UVA in the presence of 8-MOP, at different doses (J/cm²) and 3 days after irradiation.

FIG. 5 shows the delta-apoptosis (%) of JURKAT cells irradiated with UVB at different doses (J/cm²), 1 and 2 days after irradiation.

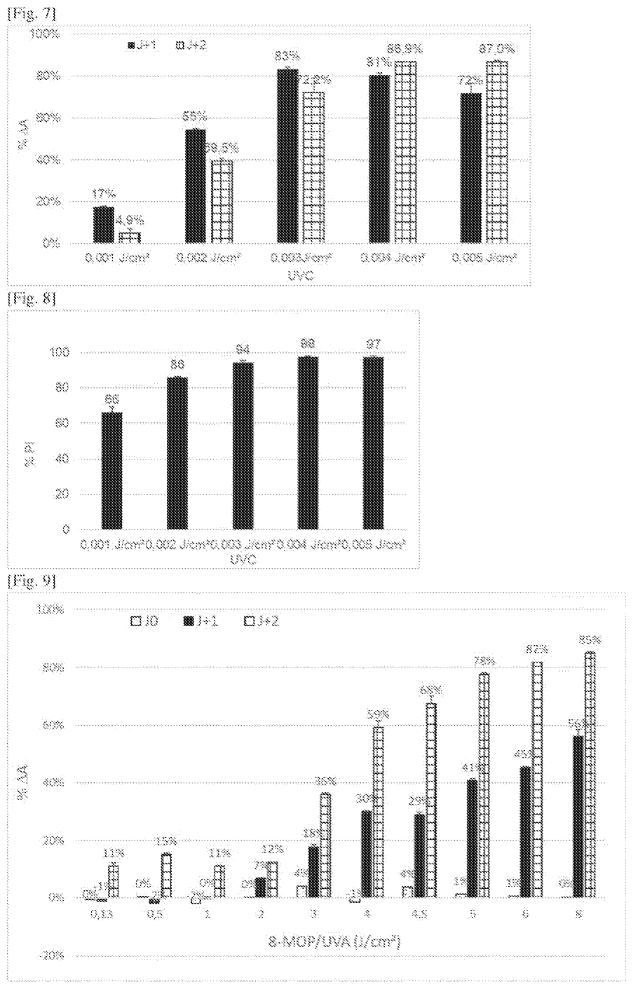

FIG. 6 shows the inhibition of proliferation (%) of JURKAT cells irradiated with UVB, at different doses (J/cm²) and 3 days after irradiation.

FIG. 7 shows the delta-apoptosis (%) of JURKAT cells irradiated with UVC, 1 and 2 days after irradiation and at different doses (J/cm²).

FIG. 8 shows the inhibition of proliferation (%) of JURKAT cells irradiated with UVC, at different doses (J/cm²) and 3 days after irradiation.

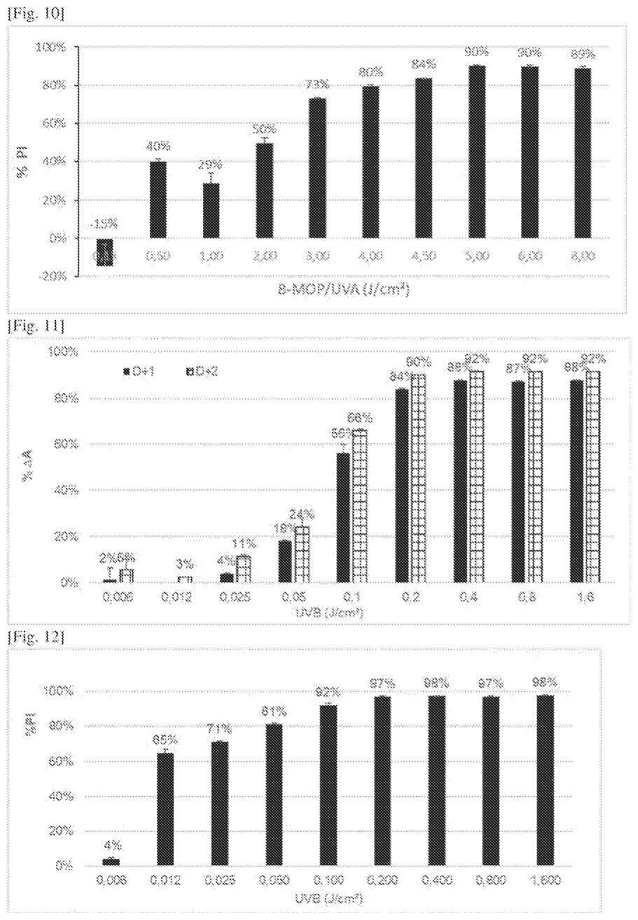

FIG. 9 shows the delta-apoptosis (%) of a suspension of JURKAT cells in plasma, irradiated with UVA in the presence of 8-MOP, at different doses (J/cm²), 0, 1 and 2 days after irradiation.

FIG. 10 shows the inhibition of proliferation (%) of a suspension of JURKAT cells in plasma, irradiated with UVA in the presence of 8-MOP, at different doses (J/cm²) and 3 days after irradiation.

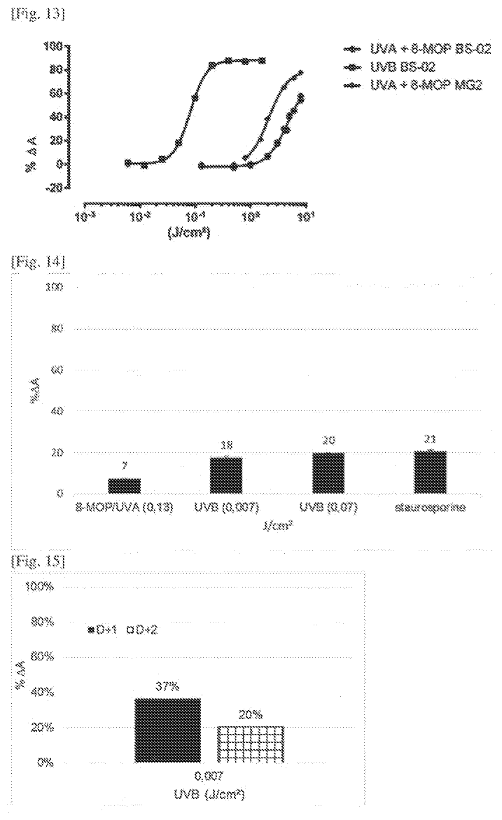

FIG. 11 shows the delta-apoptosis (%) of a suspension of JURKAT cells in plasma, irradiated with UVB at different doses (J/cm²), 1 and 2 days after irradiation.

FIG. 12 shows the inhibition of proliferation (%) of a suspension of JURKAT cells in plasma, irradiated with UVB at different doses (J/cm²) and 3 days after irradiation.

FIG. 13 shows the delta-apoptosis (%) of a suspension of JURKAT cells in plasma, irradiated with UVA in the presence of 8-MOP in two different systems or with UVB, 1 day after irradiation, according to a logarithmic scale of irradiation doses.

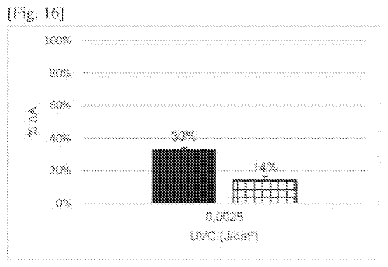

FIG. 14 shows the delta-apoptosis (%) of splenocytes irradiated with UVA in the presence of 8-MOP or irradiated with UVB at 2 different doses (J/cm²) or incubated in the presence of staurosporine—an inducer of apoptosis—1 day after irradiation or incubation.

FIG. 15 shows the delta-apoptosis (%) of splenocytes irradiated with UVB at 0.007 J/cm², 1 and 2 days after irradiation.

FIG. 16 shows the delta-apoptosis (%) of splenocytes irradiated with UVC at 2.5 mJ/cm², 1 and 2 days after irradiation.

The invention proposes a method aimed at obtaining a cellular response comparable to that obtained by conventional ECP (8-MOP/UVA), in the absence of a photoactivatable agent such as 8-MOP or porphyrin derivatives, and in particular using light alone.

According to a first aspect, the invention relates to a method for producing apoptotic mononuclear cells contained in a peripheral blood fraction from a donor. The method comprises an ultraviolet irradiation step.

Mononuclear cells (MNCs) of the peripheral blood (or sometimes referred to as “peripheral blood mononuclear cells”) are all peripheral blood cells with a single nucleus. These cells are made up of lymphocytes (T cells, B cells, NK cells) and monocytes. Erythrocytes and platelets have no nuclei, and granulocytes (neutrophils, basophils and eosinophils) have multi-lobed nuclei.

The peripheral blood fraction of a donor containing the mononuclear cells was obtained, prior to the implementation of the method of the invention, by leukapheresis, i.e. using an apheresis apparatus which takes blood from the donor, separates it by centrifugation into plasma, red blood cells and buffy coat, reinjects the plasma and red blood cells into the donor, and isolates the leukocyte-enriched fraction containing said mononuclear cells. The leukapheresis product constitutes the peripheral blood fraction containing the mononuclear cells. This leukocyte-enriched fraction has a volume of between 150 and 200 ml. It comprises more than 90% of mononuclear cells suspended in plasma.

Alternatively, the peripheral blood fraction containing the mononuclear cells has been obtained, prior to implementation of the method, by isolating the buffy coat from a whole blood sample. The whole blood sample is then centrifuged to separate the blood into plasma, red blood cells and buffy coat. The isolated buffy coat is the fraction of peripheral blood containing mononuclear cells suspended in plasma. The starting volume of whole blood is in particular between 100 and 500 ml. In the case of a whole blood sample of between 100 and 200 ml, the peripheral blood fraction containing the mononuclear cells has a volume of between 6 and 10 ml. In the case of a whole blood sample of between 450 and 500 ml, the peripheral blood fraction containing the mononuclear cells has a volume of between 40 and 50 ml.

In a variant, the peripheral blood fraction is cryopreserved before being thawed for ultraviolet irradiation. For example, mononuclear cells are stored at −80° C. in a cryopreservation solution comprising dimethyl sulphoxide (DMSO), serum albumin and hydroxyethyl starch.

In particular, the haematocrit level of the peripheral blood fraction ranges from 0 to 8%, in particular from 1 to 4%, and even more particularly is around 2%. The presence of red blood cells affects the dose of ultraviolet light adsorbed by the mononuclear cells by forming a screen for the mononuclear cells.

In order to obtain an acceptable haematocrit level, the peripheral blood fraction containing the mononuclear cells is diluted before irradiation.

Dilution is performed with plasma and/or a biocompatible solution such as a saline solution or a buffer solution. Advantageously, dilution is carried out with a saline solution only.

In one embodiment, the peripheral blood fraction has a volume of between 6 and 200 ml, and after dilution has a volume of between 200 and 500 ml, in particular in the order of 300 ml.

The plasma content of the fraction of peripheral blood containing mononuclear cells is between 30 and 50%. The presence of too much lipid and/or bilirubin in the plasma affects the clarity of the plasma, which limits the absorption of ultraviolet light by the mononuclear cells. Reducing the proportion of plasma in the peripheral blood fraction by dilution and/or centrifugation limits the risk of excessively absorbent plasma.

The fraction of peripheral blood containing the mononuclear cells and subjected to ultraviolet irradiation is devoid of photoactive agents or apoptosis-inducing agents such as methylprednisolone. The absence of such agents makes the method simpler and safer than conventional ECP, since once the peripheral blood fraction to be irradiated has been obtained, the method can be carried out entirely in a closed system.

According to one embodiment, the method is autologous, i.e. the peripheral blood fraction containing the mononuclear cells is obtained from the patient to be treated by said apoptosis-induced mononuclear cells. The donor and the patient are therefore the same person.

The method of the invention is carried out extracorporeally, once the peripheral blood fraction has been isolated from the whole blood. The peripheral blood fraction is placed in a container, separated, and isolated from the donor.

The method according to the first aspect of the invention comprises the step of subjecting said blood fraction to ultraviolet irradiation having a wavelength of between 200 nm and 320 nm.

In particular, the ultraviolet irradiation has a wavelength of between 200 and 280 nm, in particular 255 nm. Alternatively, the ultraviolet irradiation has a wavelength of between 280 and 320 nm, in particular in the order of 310 nm.

In one embodiment, ultraviolet irradiation of the peripheral blood fraction is carried out as a continuous flow during the continuous flow of the peripheral blood fraction in a tube or bag forming a serpentine flow path.

In another embodiment, ultraviolet irradiation of the peripheral blood fraction is carried out discontinuously with the peripheral blood fraction present in a container.

In order to obtain homogeneous irradiation of the mononuclear cells in the peripheral blood fraction, the peripheral blood fraction is agitated during the irradiation.

According to the method of the invention, the ultraviolet irradiation is arranged to induce a difference between the rate of apoptosis of irradiated cells and that of non-irradiated cells of greater than 15%, 48 hours after irradiation, in particular 24 hours after irradiation.

The difference between the rate of apoptosis of irradiated cells and that of non-irradiated cells is called delta-apoptosis. Delta-apoptosis is determined by calculating the difference between the apoptosis rate of irradiated cells and the apoptosis rate of non-irradiated cells.

In particular, delta-apoptosis was greater than 15% 24 hours after irradiation.

With such ultraviolet irradiation having a wavelength between 200 and 320 nm, and more particularly between 280 and 320 nm, it is possible to obtain apoptotic mononuclear cells capable of modulating the immune response in a patient.

The study of apoptosis or programmed cell death is generally carried out using double labelling of cells with annexin-V coupled to a fluorophore FITC and propidium iodide (PI). The labelled cells are analysed using flow cytometry.

Annexin-V is a protein with a high affinity for phosphatidylserines, the membrane proteins of cells. When the cell is viable, the membrane phosphatidylserines are located on the intracellular side, which prevents annexin-V from binding. As soon as the cell enters apoptosis, these proteins externalise and are expressed on both sides of the membrane. The apoptotic cells are therefore marked by annexin-V.

The combined use of PI, a DNA intercalating agent, allows apoptotic cells to be distinguished from necrotic cells. In fact, cell necrosis is accompanied by the loss of membrane integrity, which is not the case for a cell undergoing apoptosis. This allows the PI to penetrate the cell and intercalate with the DNA. This double labelling makes it possible to distinguish between viable, necrotic and apoptotic cells: viable cells are negative for PI and negative for annexin V, necrotic cells are positive for PI and negative for annexin V, early apoptotic cells are negative for PI and positive for annexin V, and late apoptotic cells are positive for PI and positive for annexin V.

Flow cytometry is a technique used to analyse the physical and biological characteristics of each cell and therefore to differentiate between the cell populations in a suspension (Carmaux, 2008). Morphological analysis allows cell populations to be distinguished according to two parameters: size (Forward scatter FSC) and granularity (Side scatter SSC). The result is a scatter plot where each point corresponds to one cell. Dense, homogeneous zones correspond to a population, and these zones can then be delimited using software such as Accuri. This allows the desired cell population to be identified and analysed.

It is advantageous that the irradiated mononuclear cells continue to undergo apoptosis when reinjected into the patient. Thus, irradiation is arranged to induce a delta-apoptosis 48 hours after irradiation greater than the delta-apoptosis 24 hours after irradiation.

To obtain an immunomodulatory effect in the patient, the mononuclear cells are in a state of apoptosis and not necrosis. Thus, in the method of the invention, irradiation is designed to induce a necrosis rate of less than 5%, in particular less than 1%.

Another effect of ECP on cells is the inhibition of cell proliferation, particularly of T lymphocytes.

In the method according to the first aspect, the irradiation is in particular arranged to further induce a rate of inhibition of cell proliferation greater than 70% three days after irradiation. In particular, the rate of inhibition of cell proliferation is greater than 90% three days after irradiation.

The rate of inhibition of cell proliferation is determined by dividing the difference between the proliferation rate of the unirradiated control cells and the proliferation rate of the irradiated cells by the proliferation rate of the unirradiated control cells, the rate of cell proliferation being determined by dividing the total number of final cells by the total number of starting cells. The rate of inhibition of proliferation was determined 3 days after irradiation and after the cells had been cultured.

The dose of ultraviolet light that needs to be delivered to the mononuclear cells to achieve the appropriate delta-apoptosis depends on many factors. These factors are intrinsic, relating to the composition of the peripheral blood fraction, such as haematocrit, plasma quantity and plasma clarity, and extrinsic, relating to the system used for irradiation, such as the shape and transparency to ultraviolet light of the irradiation container receiving the peripheral blood fraction, the configuration of the ultraviolet light source (irradiation of one or both sides of the irradiation container), the type and speed of agitation of the irradiation container and the thickness of the peripheral blood fraction in the irradiation container.

In a particular embodiment, in order to determine the appropriate dose of ultraviolet light to be applied to the peripheral blood fraction containing mononuclear cells, a cell model comprising JURKAT cells is used.

JURKAT cells are an immortalised cell line of human CD4 T lymphocytes, established in the late 1970s from the blood of a fourteen-year-old boy suffering from leukaemia.

This line has the advantage of being both available and targeted by ECP since the cells are T cells. Like mononuclear cells, JURKAT cells show a progressive increase in cell death after treatment with 8-MOP and UVA irradiation (Cunderlíková, 2014; Lauhlé, 2019).

To determine the dose of ultraviolet light to be applied to the peripheral blood fraction, JURKAT cells are subjected to ultraviolet irradiation with a wavelength of between 200 and 320 nm, more particularly between 280 and 320 nm, and the dose of light sufficient to induce a delta-apoptosis of between 30 and 40% is determined 24 hours after irradiation. More particularly, the dose of light sufficient to induce a delta-apoptosis of between 60 and 70% 48 hours after irradiation is determined.

In a first embodiment, the JURKAT cells are suspended in a PBS-type saline solution.

In another embodiment, the JURKAT cells are suspended in a 2% haematocrit solution comprising plasma, in particular between 30 and 40% plasma.

The ultraviolet irradiation has a wavelength of between 280 and 320 nm, in particular 310 nm, or a wavelength of between 200 and 280 nm, in particular 255 nm.

Advantageously, the irradiation dose is determined to be sufficient to induce a rate of inhibition of JURKAT cell proliferation greater than 70% three days after irradiation.

According to another aspect, the invention relates to an irradiation apparatus for carrying out the method described above. The irradiation apparatus comprises in particular an ultraviolet light source emitting radiation having a wavelength of between 200 and 320 nm, and a control unit configured to subject the peripheral blood fraction containing the mononuclear cells to a predetermined dose of ultraviolet irradiation to induce a difference between the rate of apoptosis of the irradiated cells and that of the non-irradiated cells of greater than 15%, 48 hours after irradiation.

The irradiation apparatus and an irradiation container for receiving a peripheral blood fraction containing mononuclear cells, said irradiation container being permeable to rays having a wavelength between 200 and 320 nm, together form a system for producing apoptotic mononuclear cells contained in a peripheral blood fraction of a donor according to the method as per the first aspect of the invention.

The irradiation container is designed to receive the peripheral blood fraction containing the mononuclear cells to be irradiated. The irradiation container is particularly suitable for containing and/or transporting the fluid to be irradiated. The irradiation container is flexible or solid.

In the case of continuous flow irradiation, the irradiation container is, for example, in the form of a tube, a bag forming a serpentine flow path, or a flow cassette.

In the case of discontinuous irradiation, the irradiation container is advantageously in the form of an irradiation bag.

An example of such an irradiation bag is shown in FIG. 1. The irradiation bag 1 is made of a material permeable to ultraviolet light, such as EVA. In FIG. 1, the irradiation bag 1 comprises an inlet orifice 2 for introducing the peripheral blood fraction into the bag and an outlet orifice 3. The inlet port 2 is connected to a tube 4 ending in a spike 5. The spike 5 is intended to be connected to a source bag containing the blood fraction comprising the mononuclear cells. The irradiation bag 1 also comprises another access port 6 for introducing a dilution solution if required.

The irradiation apparatus 7 is configured to be connected to said irradiation container 1.

According to FIG. 2, the irradiation apparatus 7 is configured to be connected to an irradiation bag 1. The irradiation apparatus 7 comprises, for example, a tray 8 on which the irradiation bag 1 is intended to be placed. The central part 9 of the tray 8 is advantageously permeable to ultraviolet light with a wavelength of between 200 and 320 nm, in particular between 280 and 320 nm, so that the irradiation bag 1 can be irradiated from either side. The central part 9 of the tray 8 is made of quartz, for example.

Advantageously, the tray 8 is a shaker tray. For example, the shaker tray

performs an orbital rotation movement in order to irradiate the contents of the irradiation bag 1 evenly.

The tray 8 is rotated by a motor 10.

The irradiation apparatus 7 also comprises a source 11 of ultraviolet light emitting radiation with a wavelength of between 200 and 320 nm, in particular between 280 and 320 nm, and even more particularly in the order of 310 nm.

In FIG. 2, the light source 11 comprises a plurality of lamps emitting ultraviolet light with a wavelength of between 200 and 320 nm, in particular between 280 and 320 nm.

In FIG. 2, the irradiation apparatus 7 comprises two banks of six lamps arranged on either side of the tray 8 in order to irradiate the irradiation bag 1 from above and below.

Alternatively, the light source 11 consists of one or two banks of light-emitting diodes.

In FIG. 2, reflectors 12 are provided to reflect the light emitted by the light source 11 towards the irradiation bag.

The irradiation apparatus 7 also comprises a control unit configured to subject the peripheral blood fraction containing the mononuclear cells to a dose of ultraviolet irradiation having a wavelength of between 200 and 320 nm, and predetermined to induce a difference between the rate of apoptosis of the irradiated cells and that of the non-irradiated cells of greater than 15%, 48 hours after irradiation.

In particular, the irradiation apparatus 7 delivers to the irradiation container a dose of ultraviolet irradiation having a wavelength of between 200 and 320 nm, predetermined to induce in the mononuclear cells a delta-apoptosis of greater than 15%, 24 hours after irradiation.

In particular, the ultraviolet irradiation has a wavelength of between 280 and 320 nm. In order to control the dose of irradiation delivered to the irradiation container by the irradiation apparatus 7, the irradiation apparatus 7 comprises one or more optical sensors arranged at the light source 11. These optical sensors detect the intensity of the irradiation emitted by the light source.

The control unit is in the form of an electronic and computer system comprising, for example, a microprocessor designed to execute a command program. Execution of this program allows the control unit to control the ultraviolet light source in particular, depending for example on the signals received by the optical sensor(s). The control unit determines, for example, the irradiation time required to reach the target dose as a function of the light intensity determined by the optical sensors.

In one embodiment, the irradiation dose is predetermined using the JURKAT cell model described above.

Alternatively, the irradiation dose is determined by modelling the response of JURKAT cells in terms of delta-apoptosis using a sigmoidal numerical model. Even without irradiation, the cells will undergo apoptosis. When high doses are applied, the apoptosis rate will saturate. Between these two extremes, the function is monotonic: delta-apoptosis increases as the dose increases.

A method is now described for operating the system described above to produce apoptotic mononuclear cells contained in a peripheral blood fraction from a donor.

The method for operating said system comprises the steps of:
(a) providing an irradiation container with a donor peripheral blood fraction containing mononuclear cells,
(b) placing said irradiation container in an irradiation apparatus comprising an ultraviolet light source emitting irradiation having a wavelength between 200 and 320 nm;
(c) irradiating said irradiation container in said irradiation apparatus with said ultraviolet irradiation, said irradiation being arranged to induce a difference between the rate of apoptosis of the irradiated cells and that of the non-irradiated cells of greater than 15%, 48 hours after irradiation, in particular 24 hours after irradiation.

In particular, the ultraviolet light source has a wavelength of between 280 and 320 nm and/or the irradiation is arranged to induce a delta-apoptosis of greater than 15%, 24 hours after irradiation.

After ultraviolet irradiation according to the method of the invention, mononuclear cells are able to modulate the immune response in a patient.

The invention therefore relates to the use of apoptotic mononuclear cells obtained by the method described above for the treatment of pathologies associated with immune system dysfunction. These pathologies are, for example, cutaneous T-cell lymphomas (CTCL) including Sézary syndrome, graft versus host disease (GvHD), solid organ transplant rejection, systemic scleroderma, atopic dermatitis, psoriasis, lupus erythematosus and Crohn's disease.

In one particular embodiment, apoptotic mononuclear cells are cryopreserved after ultraviolet irradiation for later use.

### EXAMPLES

Several experiments were carried out to demonstrate the equivalence of the conventional ECP technique with 8-MOP/UVA in terms of cellular response and the method of the invention.

### 1. Cell Model

The mononuclear cells of patients are difficult to access, so a T lymphocyte line derived from human lymphoma called JURKAT was chosen as the cell model for the experiments.

### 2. Acceptance Criteria

Criteria used to validate the in vitro efficacy of a conventional ECP

(200-333 ng/ml of 8-MOP and 2 J/cm² of UVA) are the apoptosis of T lymphocytes and the inhibition of T lymphocyte proliferation.

In the case of mononuclear cells from a patient, the acceptance criteria are:
a cell proliferation inhibition rate greater than 70% three days after the ECP and
a delta-apoptosis greater than 15%, 24 hours after the ECP (Taverna, 2015).

In the JURKAT cell model, the acceptance criteria are
a rate of inhibition of cell proliferation greater than 70%, 3 days after ECP and
a delta-apoptosis greater than 30%, 24 hours after the ECP (Lauhlé, 2019).

### 3. Preparation of Cells for Irradiation

To obtain the desired quantity of JURKAT cells, they were cultured in an RPMI 1640 medium supplemented with foetal calf serum (10% v/v), 1% L-glutamine (Lonza) and 1% penicillin-streptomycin (Lonza). Cells were grown in culture flasks placed in an incubator at 37° C. and 5% CO2. The JURKAT doubling time was approximately 27 hours.

Depending on the experiments, 10.10⁶ or 400.10⁶ JURKAT cells were used per irradiation container (Petri dishes or irradiation bags).

The cells were suspended in a solution of PBS (phosphate buffer saline) or in a 2% haematocrit solution comprising 1/14 of the anticoagulant ACD-A, a 50/50 v/v mixture of NaCl and plasma. The final volume was such that the thickness of the cell suspension in the container was around 3 mm.

In the case of UVA irradiation, the cell solution also contained 200 ng/ ml or 333° ng/ml of 8-MOP.

### 3. Irradiation Equipment

Three types of irradiation equipment were used.

The first apparatus was the Macogenic G2 irradiation apparatus from Maco Pharma (France), used to irradiate a bag. It comprised six lamps delivering UVA radiation, arranged on either side of a UV-transparent quartz tray. The plate agitated the irradiation bag at a speed of 60 rpm to ensure homogeneous irradiation.

The second apparatus was the Macotronic UV irradiation apparatus from Maco Pharma (France), used to irradiate a bag. It comprised six lamps delivering UVC radiation (254 nm), arranged on either side of a quartz plate transparent to UV light. The plate agitated the irradiation bag at a speed of 110 rpm to ensure homogeneous irradiation.

The third irradiation apparatus is the BS02 hood irradiator from Opsytec Dr Gröbel used to irradiate a Petri dish. This irradiator delivered defined doses of irradiation using removable lamps that could be placed inside the apparatus. The lamps used were UVA with a peak at 352 nm, UVB with a peak at 311 nm, or UVC with a peak at 257 nm. A dosimeter controlled the irradiation time based on the irradiation dose measured by the sensor inside the apparatus. To ensure homogeneous irradiation of the cell solution, an orbital agitator was placed inside the irradiation chamber of the apparatus. The agitation speed was approximately 450 rpm.

### 4. Cell Characterisation: Apoptosis Tests and Measurement of Cell Proliferation

At the end of the irradiation step, the cell solutions with a haematocrit level of 2% are washed in order to recover as many JURKAT cells as possible for culture with low contamination by red blood cells. To do this, the components were separated by density gradient using a Ficoll solution (GE Healthcare).

After JURKAT isolation, the cells were washed three times to remove the Ficoll solution. The first two washes were performed in a PBS solution with 2 mM EDTA (Thermo Fisher), and the third was performed in a PBS solution. The cells were then re-suspended in supplemented RPMI medium and counted using Vi-cell and ABX in order to estimate the number of JURKAT that had been isolated and to assess the quantity of residual red blood cells.

To monitor the parameters of interest (apoptosis and inhibition of JURKAT proliferation), the cells were cultured using cell counting. To do this, 5.10⁶ treated cells and 5.10⁶ untreated cells were seeded in 10 ml of culture medium.

### 4.1 Apoptosis Test

Apoptosis was studied by flow cytometry, using double labelling of cells with annexin-V coupled to a fluorophore FITC and propidium iodide (PI).

Delta-apoptosis was determined by the following formula:

Δapoptosis=(% apoptosis of treated cells−% apoptosis of untreated cells) [Math. 1]

### 4.2 Inhibition of Cell Proliferation

The initial JURKAT population was 5.10⁶ cells per culture flask. The number of cells per culture flask was then counted by Vi-cell.

To do this, the culture medium with the cells was centrifuged at 1500 rpm for 5 minutes and the pellet was resuspended in supplemented RPMI culture medium. Depending on the cell density, a 1/10 dilution in PBS could be performed.

The percentage inhibition of cell proliferation on day n (Dn) was determined by the following formula:

% PI(Dn)=100−% P(Dn) [Math. 2]

in which % P(Dn) is the cell proliferation rate at n days, determined by the following formula:

$$\% P ( Jn ) = \frac{number of total cell s in the sample on Dn}{number of total control cells on D 0} \times 100 \qquad [ Math . 3 ]$$

### Example 1: Comparison of 8-MOP/UVA, UVB and UVC Treatments on JURKAT Cells in PBS

The aim of this experiment was to determine the irradiation doses for each of the radiations giving rise to (i) a rate of delta-apoptosis of JURKAT cells between 30% and 40%, one day after irradiation and 50% and 60%, two days after irradiation, and (ii) a percentage of inhibition of proliferation greater than 70%, 3 days after irradiation.

Initially, these experiments were carried out on cells suspended in PBS (without plasma or haematocrit) in order to determine the minimum irradiation dose required.

The 2 ml cell suspension containing 10.10⁶ cells was placed in a Petri dish. For UVA irradiation only, 200 ng/ml of 8-MOP were added to the cell suspension. The Petri dish was then placed in the BS02 irradiation apparatus to be irradiated with one of three irradiations: UVA, UVB or UVC. Agitation was set at 450 rpm.
a. ECP 8-MOP/UVA

For UVA irradiation, the JURKAT cell suspension contained 200 ng/ml of 8-MOP. In a first series of tests, UVA irradiation doses of between 0.03 and 0.4 J/cm² were tested. In a second series of tests and based on the results obtained with the first tests, three doses (0.1 J/cm², 0.13 J/cm² and 0.16 J/cm²) were used. The UVA dose of 0.13 J/cm² met the two acceptance criteria in terms of delta-apoptosis and inhibition of proliferation (FIG. 3 and FIG. 4).

For all the doses of UVA radiation tested, the rate of cell necrosis remained below 1%.
b. UVB Irradiation

After testing irradiation doses ranging from 0.5 mJ/cm² to 1 J/cm², the doses of interest were narrowed down to between 1 and 11 mJ/cm². The dose of 7 mJ/cm² was identified as meeting the two acceptance criteria in terms of delta-apoptosis and inhibition of proliferation (FIG. 5 and FIG. 6).

24 hours after irradiation, for an irradiation dose of 7 mJ/cm², around 20% of cells were late apoptotic and 40% were early apoptotic.

For all doses of UVB irradiation between 0.5 mJ/cm² and 1 J/cm², the rate of cell necrosis was less than 1%.
c. UVC Irradiation

After testing irradiation doses ranging from 0.5 to 3.5 mJ/cm², the doses of interest between 1 and 5 mJ/cm² were re-tested (FIG. 7 and FIG. 8) to finally test three doses (2 mJ/cm², 2.5 mJ/cm² and 3 mJ/cm²). The UVC irradiation dose of 2.5 mJ/cm² was identified as meeting both acceptance criteria in terms of delta-apoptosis and inhibition of proliferation.

24 hours after irradiation, for an irradiation dose of 2 mJ/cm², around 40% of cells were late apoptotic and 30% were early apoptotic.

It should be noted that, unlike UVA/8-MOP and UVB irradiation, delta-apoptosis did not increase between the first and second day with UVC irradiation at a dose of 2 mJ/cm².

For all the UVC doses tested, the rate of cell necrosis remained below 1%.

### Example 2: Comparison of 8-MOP/UVA and UVB Treatments on JURKAT Cells Suspended in a Plasma/Saline Solution with a Haematocrit Level of 2%

The aim of this experiment was to determine the irradiation doses for each of the radiations required to achieve the following acceptance criteria:
a delta-apoptosis of JURKAT cells of between 30% and 40% at day +1 and 50% and 60% at day +2, and
a proliferation inhibition rate of over 70% at D+3.

A 2 ml solution was prepared containing 10.10⁶ JURKAT cells at 2% haematocrit, 1/14 ACD-A, 50/50 v/v saline and plasma. For UVA irradiation only, 333 ng/ ml of 8-MOP was added.

The 2 ml cell suspension comprising 10.10⁶ cells was placed in a Petri dish, and the Petri dish was then placed in the BS02 irradiation apparatus. This apparatus was used for both UVA and UVB irradiation. Agitation was set at 450 rpm.
a. 8-MOP/UVA

In terms of delta-apoptosis and inhibition of proliferation, the acceptance criteria were reached for irradiation at 4 J/cm² for delta-apoptosis and 3 J/cm² for the rate of proliferation inhibition (FIG. 9 and FIG. 10).
b. UVB

In terms of delta-apoptosis, the acceptance criterion was reached from a dose of 0.1 J/cm², while in terms of proliferation inhibition, the acceptance criterion was reached from 0.025 J/cm² (FIG. 11 and FIG. 12).

24 hours after irradiation, for an irradiation dose of 0.1 J/cm², around 40% of cells were late apoptotic and 25% were early apoptotic.

### Example 3: UVC Treatment of JURKAT Cells Suspended in a Saline/Plasma Solution with a Haematocrit Level of 2%

In this experiment, JURKAT cells were suspended in a solution of 2% haematocrit, 1/14 ACD-A, 50/50 v/v saline and plasma. The cell suspension containing 400.10⁶ cells and having a volume of 330-400 ml was placed in a UVC-permeable bag.

Irradiation was carried out using the Macotronic UV apparatus from Macopharma (France). Two doses of 25 mJ/cm² and 50 mJ/cm² were studied.

The results showed that delta-apoptosis remains constant between the first post-irradiation day and the second post-irradiation day at a rate of around 50% for the UVC dose of 25 mJ/cm² and around 70% for the UVC dose of 50 mJ/cm².

In terms of inhibition of cell proliferation three days after irradiation, this was around 65% for the 25 mJ/cm² dose and around 80% for the 50 mJ/cm² dose.

### Example 4: Correlation with Maco Pharma's ECP Technique

Using the same matrix as in example 2, JURKAT cells (330.10⁶ cells in 300 ml, 333 ng/ml 8-MOP) were irradiated using the ECP system marketed by Maco Pharma.

The Maco Pharma ECP system consisted of a UVA-permeable bag and the Macogenic G2 irradiation apparatus designed to deliver an irradiation dose of 2 to 2.5 J/cm² depending on the haematocrit of the solution contained in the bag to be irradiated. The irradiation cycle lasted approximately 12 minutes.

Acceptance criteria in terms of delta-apoptosis and proliferation inhibition were achieved from a dose of 2 J/cm², which corresponded to the system's recommendations.

In order to model the cellular response in terms of delta-apoptosis, a sigmoidal numerical model was applicable.

FIG. 13 shows a graphical representation of the results obtained using a logarithmic scale for the irradiation doses.

Acceptance criteria depended on the combination of irradiation container and apparatus and the optical configuration of the system. For example, a dose of 2.1 J/cm² in UVA delivered with the Macogenic G2 is equivalent to a dose of 4 J/cm² with the BS02 irradiator in UVA or a dose of 0.081 J/cm² with the BS02 irradiator in UVB.

### Example 4: Murine Splenocyte Models

As part of the preparation of a study on a murine model of GvH, an experiment was carried out to check whether the doses found by irradiating JURKAT (example 1) could induce apoptosis in mouse splenocytes.

A suspension of splenocytes (Immune Insight, France) containing 5.10⁶ cells was irradiated with the BS02 irradiator at the following doses: 0.13 J/cm² for UVA/8-MOP, 7 mJ/cm² and 70 mJ/cm² for UVB, and 2.5 mJ/cm² for UVC.

In the case of UVA irradiation, the cell suspension contained 200 ng/ml of 8-MOP.

Each dose tested induced apoptosis. It should be noted that the rate of delta-apoptosis decreased 2 days after irradiation, this being due to a high rate of apoptosis measured in the control (FIG. 14 to FIG. 16).

1. A method for producing apoptotic mononuclear cells contained in a peripheral blood fraction of a donor,the peripheral blood fraction having a plasma content of between 30 and 50%, the method comprising the step of subjecting the peripheral blood fraction to ultraviolet irradiation, wherein the ultraviolet irradiation has a wavelength of between 200 nm and 320 nm, the irradiation being arranged to induce a difference between the rate of apoptosis of the irradiated cells and that of the non-irradiated cells of greater than 15%, 48 hours after irradiation, to obtain apoptotic mononuclear cells capable of modulating the immune response in a patient.

2. The method according to claim 1, wherein the irradiation is arranged to induce a difference between the rate of apoptosis of the irradiated cells and that of the non-irradiated cells of greater than 15%, 24 hours after irradiation.

3. The method according to claim 1, wherein the irradiation is arranged to further induce a rate of inhibition of cell proliferation greater than 70%, three days after irradiation.

4. The method according to claim 1, wherein the ultraviolet irradiation has a wavelength of between 280 and 320 nm, in particular 310 nm.

5. The method according to claim 1, wherein the peripheral blood fraction is obtained by leukapheresis or by isolating the buffy coat from a whole blood sample.

6. The method according to claim 1, wherein the peripheral blood fraction comprises more than 90% mononuclear cells.

7. The method according to claim 1, wherein, prior to the irradiation step, the method comprises the step of diluting the peripheral blood fraction with a saline solution.

8. The method according to claim 1, wherein the peripheral blood fraction contains a haematocrit level ranging from 0 to 8%.

9. The method according to claim 1, wherein the peripheral blood fraction has a volume of between 6 ml and 200 ml.

10. The method according to claim 1, wherein the peripheral blood fraction containing the mononuclear cells is agitated during irradiation.

11. The method according to claim 1, wherein the fraction of blood containing the mononuclear cells is devoid of photoactive agent or apoptosis-inducing agent.

12. An irradiation apparatus for carrying out the method according to claim 1, comprising an ultraviolet light source emitting radiation having a wavelength of between 200 and 320 nm, and configured to subject the peripheral blood fraction containing the mononuclear cells to a predetermined dose of ultraviolet irradiation to induce a difference between the rate of apoptosis of the irradiated cells and that of the non-irradiated cells of greater than 15%, 48 hours after irradiation.

13. The irradiation apparatus according to claim 12 further comprising:
an irradiation container configured to receive a peripheral blood fraction containing mononuclear cells, the irradiation container being permeable to rays having a wavelength of between 200 and 320 nm.

14. Apoptotic mononuclear cells obtained by the method according to claim 1, for the treatment of pathologies associated with a dysfunction of the immune system, such as cutaneous T-cell lymphoma (CTCL) including Sézary syndrome, graft versus host disease (GvHD), solid organ transplant rejection, systemic scleroderma, atopic dermatitis, psoriasis, lupus erythematosus, and Crohn's disease.

15. The irradiation apparatus as claimed in claim 12, wherein it further comprises a stirring plate and optical sensors which detect the intensity of the irradiation emitted by the light source.
